(19) United States
(12) Patent Application Publication
(10) Pub. No.: US 2007/0179359 A1
(43) Pub. Date: Aug. 2, 2007

(54) Healthy city living guide and related functionality for managing health

(76) Inventor: Amanda M. Goodwin, West Hollywood, CA (US)

Correspondence Address: PERKINS COIE LLP;PATENT-SEA, P.O. BOX 1247, SEATTLE, WA, 98111-1247, US

(21) Appl. No.: 11/651,924

(22) Filed: Jan. 9, 2007

Related U.S. Application Data
(60) Provisional application No. 60/757,939, filed on Jan. 10, 2006.
(60) Provisional application No. 60/862,859, filed on Oct. 25, 2006.

(51) Int. Cl. A61B 5/00; A63B 71/00

(52) U.S. Cl. 600/300; 128/921; 482/8

(57) ABSTRACT

Techniques are described for managing a user's health. In various embodiments, these techniques include a receiving a caloric request and a resting metabolic rate, computing an expended number of calories based on the user's resting metabolic rate and physical activity performed by the user, computing a consumed number of calories based on food the user consumes, determining a status for the user based on whether the user is to consume calories or expend calories, and sending an alert to the user.

### CROSS-REFERENCE TO RELATED APPLICATION(S)

This application claims the benefit of U.S. Provisional Patent Application No. 60/757,939, entitled “CELL PHONE BASED CALORIE COUNTER,” filed on Jan. 10, 2006, and U.S. Provisional Patent Application No. 60/862,859, entitled “HEALTHY CITY LIVING GUIDE AND RELATED FUNCTIONALITY FOR MANAGING HEALTH,” filed on Oct. 25, 2006, each of which is hereby incorporated by reference in its entirety.

### BACKGROUND

Many studies show that people are becoming obese. Sedentary lifestyles, availability of unhealthy foods, and increased food portions have contributed to obesity, which many believe is caused by consuming more calories than is expended. People who are gaining weight employ various techniques to ward off obesity, such as exercising, dieting, and consuming healthier foods and, dietary supplements.

However, these techniques can sometimes be difficult to employ. As an example, during a busy day, people may forget to exercise or count caloric intake. As another example, people who are traveling may be unable to easily locate activity centers or food sources that help them to manage their health. Often, people lack the motivation to live healthy lives.

### BRIEF DESCRIPTION OF THE DRAWINGS

The present invention is illustrated in an exemplary manner by the accompanying drawings. The drawings should be understood as exemplary rather than limiting, as the scope of the invention is defined by the claims.

FIG. 1 illustrates an embodiment of a health guidance or calorie counting system.

FIG. 2 illustrates an embodiment of a user interface of an embodiment of a health guidance or calorie counting system.

FIG. 3 illustrates an embodiment of a process of counting calories.

FIG. 4 illustrates an embodiment of a process of alerting a user to eat.

FIG. 5 illustrates an embodiment of a machine-readable medium embodying a server part of a health guidance or calorie counting system.

FIG. 6 illustrates an embodiment of a machine-readable medium embodying a client part of a health guidance or calorie counting system.

FIG. 7 illustrates an embodiment of a network which may be used with a health guidance or calorie counting system.

FIG. 8 illustrates an embodiment of a computer or machine which may be used with a health guidance or calorie counting system.

FIG. 9 illustrates an example of a cell phone with a display screen for displaying calories consumed and an icon for readily accessing health guidance and calorie counting functionality.

### DETAILED DESCRIPTION

Various health-related programs and devices are available to users who want to improve their health. Conventionally, these offer a focused set of features that address a single aspect of a user's health management needs. For example, a dieter may be able to access an electronic food nutrition reference, or a listing of local health-related businesses. Some may even be available in a convenient form-factor, such as a handheld device. These offerings, however, do not adequately meet the needs of users desiring optimal health.

Systems, methods and apparatuses are disclosed for providing to users integrated sets of health advising and monitoring features and Healthy Living City Guides (“the health system” or simply “the system”). The health system can use a handheld computing device to provide real-time feedback based on a user's choices and flexible, timely reminders for the user. A user can employ the health system to access nutritional values databases to determine calories associated with food the user will consume and accumulate daily nutritional totals. Users can also employ the health system to provide real-time feedback about how the consumed food affects their nutritional goals. The health system can also provide diet menus or other specialized menus, such as for marathon runners, elite athletes, or for people who have specific health concerns they want to address through food choices. The health system can also provide links to relevant health news and other fitness content, such as workout, motivational, and instructional content, such as audio/video or other multimedia instructional content. Many features provided by the health system integrate location awareness with deep and broad online references. For example, the Healthy Living City Guide can give users access to various health-related, location-specific information. The Healthy Living City Guide enables users to access geographically relevant sites local to the current location of the handheld computing device, such as lists of local restaurants serving healthy food, health food stores, gyms, parks, nature walks, health care providers, advisors, therapists, massage therapists, spas, and other health related services. The Healthy Living City Guide also enables users to access information about geographically relevant events, such as an upcoming yoga retreat in the area, a nearby health-related lecture, a sale occurring at a natural foods store, etc. The Healthy Living City Guide offers users information users can employ to manage all aspects of their health, whether they are in their home city or are traveling to an unfamiliar city, and it does this in a language that is familiar to them and in a format that is easy to use.

In some embodiments, the health system provides a calorie counting system useful with cell phones and similar handheld computing devices. A server associated with the health system in these embodiments implements a database interface, a menu information interface, a third party communication interface, an advertising interface, and a client communication interface. The database interface provides access to a database of food information. The menu interface provides access to additional food information from outside data sources. The third party communication interface provides communication with third parties, such as health professionals. The advertising interface provides access to advertisements. The client communication interface provides communications with users, such as via a cell phone or other handheld computing device.

In some embodiments, the health system includes a client module. The client module may include a user interface, communications interface, and a control module. The user interface may enable user input and output. The communications interface may interact with the client communications interface of the server. The control module may control the communications interface and the user interface. In some embodiments, the health system includes a process for receiving a user food selection at a client personal device, such as a cell phone or other handheld computing device. The process can further include looking up information related to the food selection at a server. The process can also include calculating food intake based on the food selection and personal information. The process may further include accepting alert requests. The process may also include alerting a user in response to an alert schedule. The user may choose an alert schedule for taking a specific medicine, dietary supplement, or drinking water. The user may desire an alert to occur when the user meets a specific dietary goal, such as when her cumulative caloric intake and output for the day has reached a point at which she is on track to meet a weight loss goal, or when the user needs to change her intake of a particular vitamin, mineral, or other nutrient. As an example, when advanced bio-sensors are available to monitor a user's current internal nutritional status, the health system could create alerts based on these conditions.

In various embodiments, the health system may send alerts based on various information it collects. As examples, the health system may review a user's caloric intake and activity and send the user an alert to increase physical activity. The alert may provide relevant motivational messages, reminders, news, etc. As an example, if the user has indicated a particular training regimen and the health system detects that the indicated training regimen is not being followed, the health system may send a motivational message to the user.

A user's health care provider may set or adjust an alert schedule based on information the user has provided about her diet and exercise. For example, if the user has repeatedly consumed excess calories over a particular time period, the health care provider can adjust alerts pertaining to the user's workout or meal schedule. The user's health care provider may also use this feature to enter an alert to notify the user to schedule a follow up appointment if, for example, the user has poorly managed her blood sugar. The blood sugar information can be obtained, for example, through an implanted blood sugar device or common strip-type device that communicates with the user's handheld computing device, such as a cell phone. Users may choose an alert schedule to help them manage their sleeping habits, for example, by reminding them that it is time to go to sleep.

A user may even choose to integrate a reminder with media content. For example, in addition to an alert notifying the user that it is time for her workout, the alert may provide a link to the chosen workout. When the user is ready to proceed, she clicks on the link that begins the chosen workout. The user may choose the workout or, allow a personal trainer or other health professional to create or choose a workout for her. By using this feature, the user can obtain some of the benefits of attending an instructor-led class without having to be physically present at a specific place and time. Users can also create alerts to manage other health-related goals.

In some embodiments, a cell phone-based calorie counting system is provided that includes a client-side user interface that may be used on a cell phone (or other device, such as a handheld or other computing device) to enter data and receive alerts. The health system also includes one or more of the server-side components. Between the server and client, a database of foods is provided. The database includes information on specific foods, such as a description, nutritional information, and sources from which the foods may be obtained. Thus, a McDonald's® hamburger may be included, along with nutritional information. Similarly, a Starbucks® latte with nutritional information may also be included.

Additionally, a module useful for communication with service providers or healthcare professionals may also be provided, allowing for transmission of information about a person's eating habits or recent food consumption. Moreover, advertisements may be incorporated into the health system for local health-related businesses listed in the Healthy Living City Guide, and may be displayed to users as part of the health system. Also, the health system includes links to sources of nutritional information for food and beverages which are generally available for purchase. The specific embodiments described in this document represent exemplary instances of the present invention, and are illustrative in nature rather than restrictive.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this Detailed Description section.

Reference in the specification to “one embodiment” or “an embodiment” means that a particular feature, structure, or characteristic described in connection with the embodiment is included in at least one embodiment of the invention. The appearances of the phrases “in one embodiment” or “in some embodiments” in various places in the specification are not necessarily all referring to the same embodiment, nor are separate or alternative embodiments mutually exclusive of other embodiments.

Various embodiments may be used in a cell phone based calorie counter. These embodiments may include processes, systems, or apparatuses, for example. Moreover, cell phones or other personal devices may be used. Indeed, those skilled in the relevant art will appreciate that the invention can be practiced with other communications, data processing, or computer system configurations, including: Internet appliances, hand-held devices (including personal digital assistants (PDAs)), wearable computers, all manner of cellular or mobile phones, multi-processor systems, microprocessor-based or programmable consumer electronics, set-top boxes, laptop computers, and the like. Indeed, the terms “computer,” “cell phone,” and the like are generally used interchangeably herein, and refer to any of the above devices and systems, as well as any data processor.

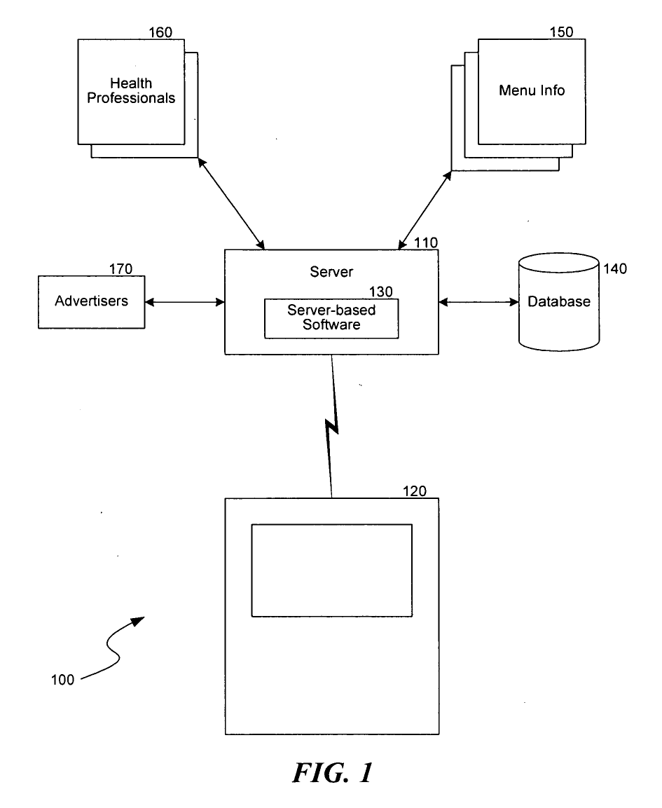

FIG. 1 illustrates one embodiment of a calorie counting system 100 that includes a server 110 and a client or personal device 120 (e.g., a cell phone). Server 110 includes server based software 130. Software module 130 interfaces with database 140, menu information 150, health professionals 160 and advertisers 170. As described in detail below, a user may thus use personal device 120 to access nutrition information from database 140 through software module 130 and thereby track food consumption.

Database 140 includes information about food and beverages which may be selected by a user, representing the user's food intake. Menu information 150 represents sources of information about menus at restaurants and stores, including descriptions and nutritional information. Many chain restaurants publish such information, and thus it may be readily collected and consolidated in a database. Database 140 may also include nutritional information for common dishes, food items, or beverages for the user's convenience in case nutritional information is not available for an eating establishment she has chosen. Health professionals 160 represent actual health professionals and related service providers, such as doctors, nutritionists, personal trainers and other people requested to monitor food intake. Advertisers 170 represent sources of advertising which may be provided to users for revenue generation.

Nutrition information may include calories, various components such as fat, carbohydrates, sugars, protein, starch, or other measures of food content. Thus, a user may collect and monitor intake in a variety of different areas, and may display information about various ingredients of food. This may be true regardless of whether various processes or systems are used, for example. Additionally, consumption may be monitored based on personal characteristics such as weight, body mass index (BMI), lifestyle (sedentary, active, for example).

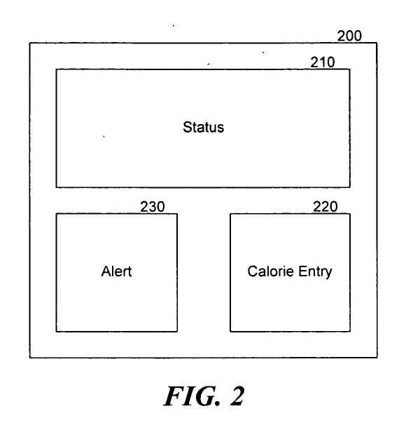

For a user to access a system such as system 100, a user interface may be utilized in a personal device. FIG. 2 illustrates an embodiment of a user interface of an embodiment of a calorie counting system. User interface 200 includes status information, a food entry option and an alert indicator. User interface 200 is representative of various user interfaces which may be implemented for use with various embodiments of systems for calorie counting.

Status indicator 210 may provide information about food intake—such as intake for the current day, calories burned for the current day, or other information. Calorie input 220 allows for entry of a new food event—consumption of food or beverages. Entry may be done based on type of food eaten, or based on an ingredient consumed such as fat or protein, for example. Alert 230 may provide an alert or indication that it is time to eat. This alert may be based on a schedule entered by a user, a user's health care provider, a user's subscription to alert schedules with specific goals (such as a 2 times a day, 10 minute workout program), or a separate programmer. Thus, a user may review current status, enter a new food event (consumption) or receive an alert to proceed to eat.

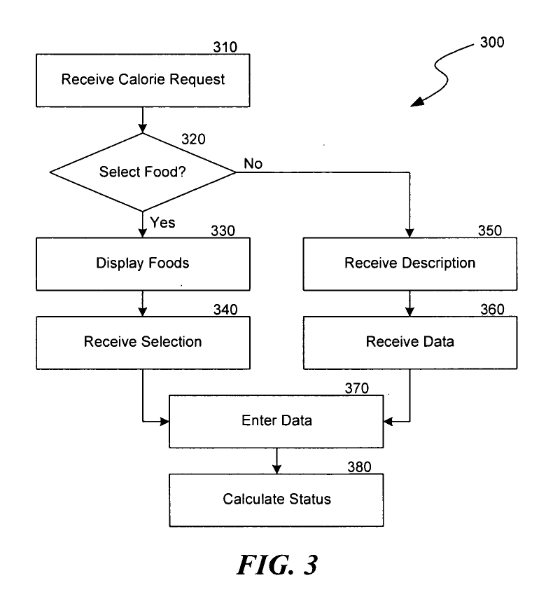

Various processes may be used to implement a system such as system 100 or other systems of calorie counting. FIG. 3 illustrates an embodiment of a process of counting calories. Process 300 includes receiving a calorie request, determining if a food is to be selected or an ingredient is to be reported, displaying foods and receiving a selection or receiving a description and nutrition data, entering the data into a counter and calculating current status. Process 300 and other processes of this document are implemented as a set of modules, which may be process modules or operations, software modules with associated functions or effects, hardware modules designed to fulfill the process operations, or some combination of the various types of modules, for example. The modules of process 300 and other processes described herein may be rearranged, such as in a parallel or serial fashion, and may be reordered, combined, or subdivided in various embodiments.

A calories request is received at module 310, indicating food has been consumed. Thus, the user desires to record food/beverage (food is used to refer to foods or beverages in this document) consumption. At module 320, the user selects either the option of choosing a predefined food or entering specific information. If a food selection is chosen, at module 330, available foods are displayed, such as in a menu or drop-down list, for example. A selection is then received at module 340, thereby implying not only the food eaten, but related information about calories, fat content, content of specific types of fat, vitamins, and other ingredient information. Data is then entered as the latest food consumed at module 370.

Alternatively, a specific food need not be selected. Instead, at module 350, a description of a specific type or portion of food may be entered. Additionally, at module 360, nutrition information for the food is entered. Such nutrition information may be entered at the user's option, and may include fat, protein, sugar, carbohydrate, fiber, and other ingredient measures. The entered data is then provided for calculation at module 370.

Information provided at module 370 is calculated into a current status at module 380. This provides an indication of what a user has consumed in a given day, for example. Such data may then be displayed to the user, or communicated to a third party, for example.

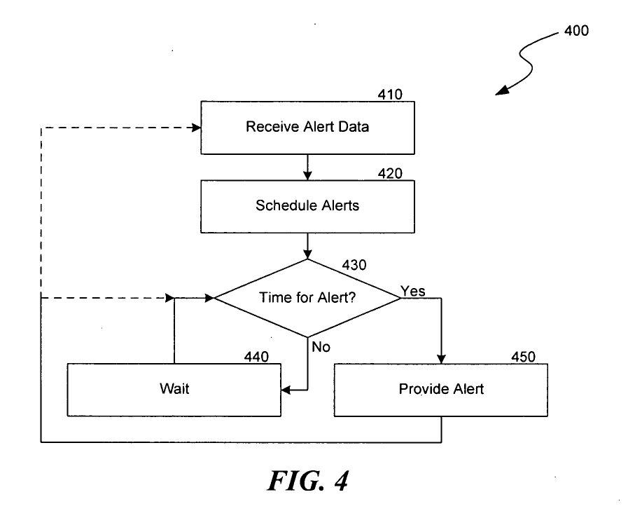

In addition to counting calories, a user may be alerted to desired times at which to eat. Such alerts may be provided responsive to a schedule entered by the user, for example. FIG. 4 illustrates an embodiment of a process of alerting a user to eat. Process 400 includes receiving alert data, scheduling alert(s), determining an alert is due, and alerting the user.

Process 400 initiates with receipt of alert data at module 410. Such alert data may be times for alerts, or relative increments of time for eating intervals. Based on alert data of module 410, alerts are scheduled at module 420 (an alert schedule is assembled). At module 430, a determination is made as to whether an alert is scheduled for the current time. If not, the process waits at module 440. If so, an alert is provided, such as by sounding an alert sound (a chime for example), displaying an alert in a user interface, or otherwise activating a perceptible alert at module 450. The process then returns to either determining if more alerts are scheduled at module 430 or receiving alert data at module 410.

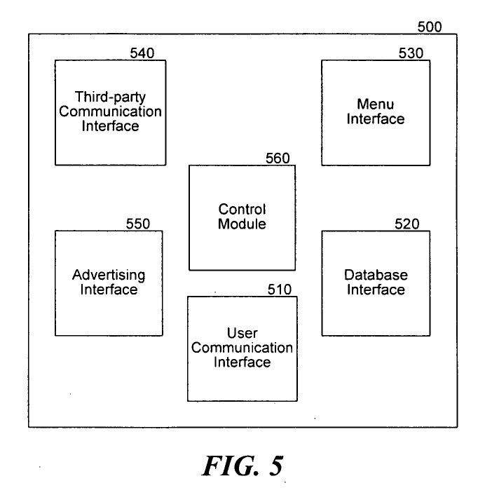

Processes may be implemented by machines responsive to instructions executed by a processor. The instructions may be embodied in a machine-readable medium, such that the medium embodies software modules. FIG. 5 illustrates an embodiment of a machine-readable medium embodying a server part of a calorie counting and health guidance system. The medium 500 embodies a database interface, menu information interface, third party communication interface, an advertising interface and a client communication interface and a control module.

The database interface 520 provides for access to a database of food information. The menu interface 530 provides for access to additional food information from outside data sources (e.g. websites). The third party communication interface 540 provides for communication with third parties such as health professionals, personal trainers, and other people the user wishes to inform about eating information. The advertising interface 550 provides for access to advertisements, such as from advertising distributors for example. Advertisements provided as part of the Healthy Living City Guide may highlight special offers from health-related business close to the mobile phone's location, contain coupons or discounts, and provide timely information, such as the availability of a massage appointment from a massage therapist located near the mobile phone's present location. The health system may feature companies participating in the Healthy Living City Guide more prominently than those who simply purchase advertising, or it may rank listings based on additional fees paid or on user feedback. The client communication interface 510 provides for communication with a client such as a process on a cell phone or personal device. A control module 560 is also provided to control the other modules.

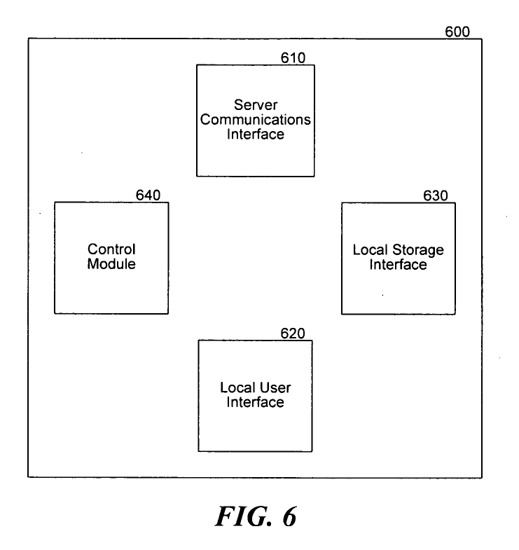

Similarly, a machine-readable medium may embody components of a client software module. FIG. 6 illustrates an embodiment of a machine-readable medium embodying a client part of a calorie counting system. Medium 600 includes a user interface, communications interface, a local storage interface, and a control module. The user interface 620 may allow for user input and output. The communications interface 610 may interact with a client communications interface of a server. The local storage interface 630 may provide access to local storage on a client device such as a personal device. The control module 640 may control the communications interface 610, local storage interface 630 and the user interface 620.

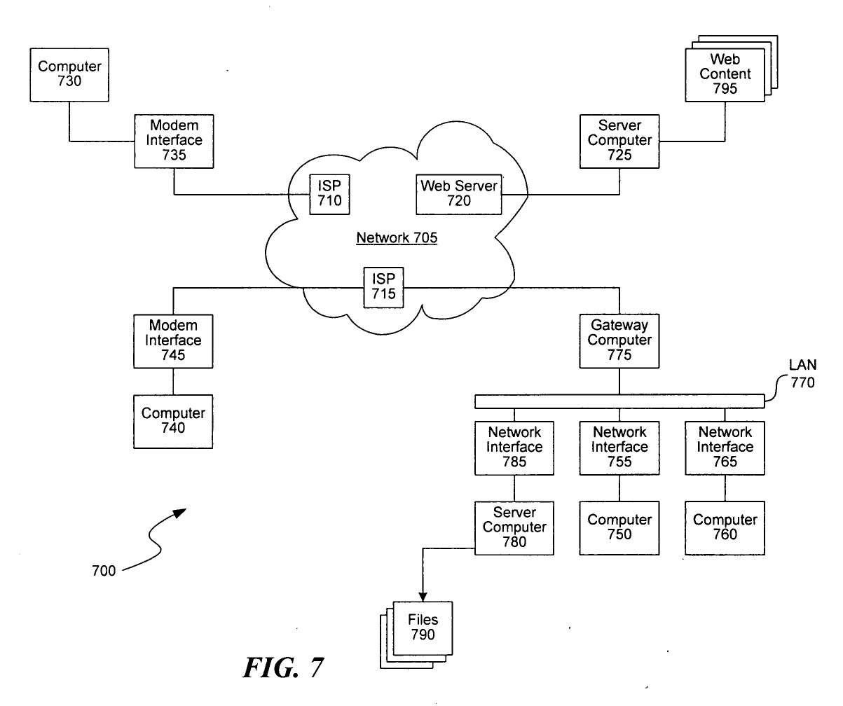

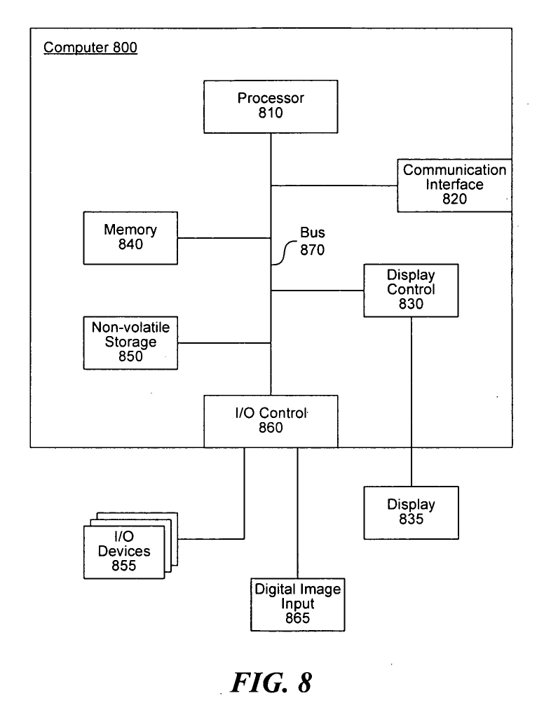

The following description of FIGS. 7-8 is intended to provide an overview of computer hardware and other operating components suitable for performing the methods of the invention described above and hereafter, but is not intended to limit the applicable environments. Similarly, the computer hardware and other operating components may be suitable as part of the apparatuses of the invention described above. The invention can be practiced with other computer system configurations, including hand-held devices, multiprocessor systems, microprocessor-based or programmable consumer electronics, network PCs, minicomputers, mainframe computers, and the like. The invention can also be practiced in distributed computing environments where tasks are performed by remote processing devices that are linked through a communications network.

FIG. 7 shows several computer systems that are coupled together through a network 705, such as the Internet. The term “Internet” as used herein refers to a network of networks which uses certain protocols, such as the TCP/IP protocol, and possibly other protocols such as the hypertext transfer protocol (HTTP) for hypertext markup language (HTML) documents that make up the World Wide Web (web). The physical connections of the Internet and the protocols and communication procedures of the Internet are well known to those of skill in the art.

Access to the Internet 705 is typically provided by Internet service providers (ISP), such as the ISPs 710 and 715, or through wireless service providers. Users on client systems, such as client computer systems 730, 740, 750, and 760 obtain access to the Internet through the Internet service providers, such as ISPs 710 and 715. Access to the Internet allows users of the client computer systems to exchange information, receive and send e-mails, and view documents, such as documents which have been prepared in the HTML format. These documents are often provided by web servers, such as web server 720 which is considered to be “on” the Internet. Often these web servers are provided by the ISPs, such as ISP 710, although a computer system can be set up and connected to the Internet without that system also being an ISP.

The web server 720 is typically at least one computer system which operates as a server computer system and is configured to operate with the protocols of the World Wide Web and is coupled to the Internet. Optionally, the web server 720 can be part of an ISP which provides access to the Internet for client systems. The web server 720 is shown coupled to the server computer system 725 which itself is coupled to web content 795, which can be considered a form of a media database. While two computer systems 720 and 725 are shown in FIG. 7, the web server system 720 and the server computer system 725 can be one computer system having different software components providing the web server functionality and the server functionality provided by the server computer system 725 which will be described further below.

Client computer systems 730, 740, 750, and 760 can each, with the appropriate web browsing software, view HTML pages provided by the web server 720. The ISP 710 provides Internet connectivity to the client computer system 730 through the modem interface 735 which can be considered part of the client computer system 730. The client computer system can be a personal computer system, a network computer, a web TV system, or other such computer system.

Similarly, the ISP 715 provides Internet connectivity for client systems 740, 750, and 760, although as shown in FIG. 7, the connections are not the same for these three computer systems. Client computer system 740 is coupled through a modem interface 745 while client computer systems 750 and 760 are part of a LAN. While FIG. 7 shows the interfaces 735 and 745 as generically as a “modem,” each of these interfaces can be an analog modem, isdn modem, cable modem, satellite transmission interface (e.g. “direct PC”), or other interfaces for coupling a computer system to other computer systems.

Client computer systems 750 and 760 are coupled to a LAN 770 through network interfaces 755 and 765, which can be Ethernet network or other network interfaces. The LAN 770 is also coupled to a gateway computer system 775 which can provide firewall and other Internet related services for the local area network. This gateway computer system 775 is coupled to the ISP 715 to provide Internet connectivity to the client computer systems 750 and 760. The gateway computer system 775 can be a conventional server computer system. Also, the web server system 720 can be a conventional server computer system.

Alternatively, a server computer system 780 can be directly coupled to the LAN 770 through a network interface 785 to provide files 790 and other services to the clients 750, 760, without the need to connect to the Internet through the gateway system 775.

FIG. 8 shows one example of a conventional computer system that can be used as a client computer system or a server computer system or as a web server system. (Such a computer system also represents many of the functions performed by an Internet service provider, such as ISP 710.) The computer system 800 interfaces to external systems through the modem or network interface 820, Which may be a wireless transceiver. It will be appreciated that the modem or network interface 820 can be considered part of the computer system 800. This interface 820 can be an analog modem, ISDN modem, cable modem, token ring interface, satellite transmission interface (e.g. “direct PC”), or other interfaces for coupling a computer system to other computer systems.

The computer system 800 includes a processor 810, which can be a conventional microprocessor such as an Intel® Pentium microprocessor or Motorola® PowerPC microprocessor. Memory 840 is coupled to the processor 810 by a bus 870. Memory 840 can be dynamic random access memory (dram) and can also include static ram (SRAM). The bus 870 couples the processor 810 to the memory 840, also to non-volatile storage 850, to display controller 830, and to the input/output (I/O) controller 860.

The display controller 830 controls in the conventional manner a display on a display device 835 which can be a cathode ray tube (CRT) or liquid crystal display (LCD). The input/output devices 855 can include a keyboard, disk drives, printers, a scanner, and other input and output devices, including a mouse or other pointing device. The display controller 830 and the I/O controller 860 can be implemented with conventional well known technology. A digital image input device 865 can be a digital camera which is coupled to an I/O controller 860 in order to allow images from the digital camera to be input into the computer system 800.

The non-volatile storage 850 is often a magnetic hard disk, an optical disk, or another form of storage for large amounts of data. Some of this data is often written, by a direct memory access process, into memory 840 during execution of software in the computer system 800. One of skill in the art will immediately recognize that the terms “machine-readable medium” or “computer-readable medium” includes any type of storage device that is accessible by the processor 810 and also encompasses a carrier wave that encodes a data signal.

The computer system 800 is one example of many possible computer systems which have different architectures. For example, personal computers based on an Intel microprocessor often have multiple buses, one of which can be an input/output (I/O) bus for the peripherals and one that directly connects the processor 810 and the memory 840 (often referred to as a memory bus). The buses are connected together through bridge components that perform any necessary translation due to differing bus protocols.

Network computers are another type of computer system that can be used with the present invention. Network computers do not usually include a hard disk or other mass storage, and the executable programs are loaded from a network connection into the memory 840 for execution by the processor 810. A Web TV system, which is known in the art, is also considered to be a computer system according to the present invention, but it may lack some of the features shown in FIG. 8, such as certain input or output devices. A typical computer system will usually include at least a processor, memory, and a bus coupling the memory to the processor.

In addition, the computer system 800 is controlled by operating system software which includes a file management system, such as a disk operating system, which is part of the operating system software. One example of an operating system software with its associated file management system software is the family of operating systems known as Windows® from Microsoft Corporation of Redmond, Wash., and their associated file management systems. Another example of an operating system software with its associated file management system software is the Linux operating system and its associated file management system. The file management system is typically stored in the non-volatile storage 850 and causes the processor 810 to execute the various acts required by the operating system to input and output data and to store data in memory, including storing files on the non-volatile storage 850.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention, in some embodiments, also relates to apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus. Aspects of the invention can also be embodied in a special purpose computer or data processor that is specifically programmed, configured, or constructed to perform one or more of the computer-executable instructions explained in detail herein.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language, and various embodiments may thus be implemented using a variety of programming languages.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the invention provided herein can be applied to other systems, not necessarily the health system described above. The elements and acts of the various embodiments described above can be combined to provide further embodiments. Any patents and applications and other references noted above, including any that may be listed in accompanying filing papers, are incorporated herein by reference. Aspects of the invention can be modified, if necessary, to employ the health systems, functions, and concepts of the various references described above to provide yet further embodiments of the invention.

Furthermore, the invention, in various embodiments, may include one or more of the following features as discussed below. One such feature is a simple menu interface. For example, the interface may allow a user to select a food by narrowing it to a geographical area and then a restaurant, at which time the food is selected from a scrolling menu of items on the restaurant menu.

Additionally, the interface may provide a workout timer. This timer may be provided in a variety of ways. For example, a simple countdown timer may be set by the user. Alternatively, a user may select a desired workout activity (e.g. walking, running, rollerblading) and the health system may calculate how much time the user needs to spend on that activity to reach a desired goal (and then set a timer). The desired goal may be user input, or based on overall information such as a user selecting a plan to lose a predetermined amount of weight, and the health system then operating from that plan. Similarly, the health system may calculate the amount of time based on what was recently eaten, and the time/energy required to “burn off” the recently eaten food.

Similarly, the health system may be integrated with exercise equipment through various links and sensors. Thus, the health system may monitor activity on exercise equipment (e.g. the treadmill is in use) through a wired or wireless (e.g. Bluetooth or other short range wireless protocol) connection, for example. Similarly, the health system may monitor sensors on the exercise equipment, such as a heart rate monitor for example. Alternatively, sensors separate from the exercise equipment may be monitored, such as blood oxygen level sensors, independent heart rate monitors, or other physiological conditions. Additionally, sensors such as pedometers may be monitored during exercise, whether the exercise is tied to a machine or out in open space. Various biometric sensors and coupling technologies may be used to accomplish this.

Additionally, a user may enter her resting metabolic rate (RMR) if the user has this information, or the user can enter information for the health system to determine an RMR for the user. Optionally, the health system may make use of several metabolic rates that the user may enter or select. As the user exercises and develops more lean muscle mass, she may want to periodically adjust her RMR to assure that she is using accurate information to manage her caloric intake. The health system can then use a metabolic rate, for example, to give real-time feedback to a user about her caloric balance for the day. Even while resting, people use caloric energy to maintain normal body functioning. As a result, an accurate caloric balance should include the number of calories burned while at rest. Measuring from midnight, for example, a user may have used 480 calories by simply sleeping until 8:00 a.m. (60 RMR calories per hour for 8 hours). The user may wake up and eat a breakfast of a banana and a bowl of cereal that contains, for example, 600 calories. The status would show that the user is 120 calories ahead of her needs. If she then performs some physical activity that consumes 50 calories, the status would show that she is only ahead by 70 calories. If she does not eat anything until noon, the monitor will show that she is now 170 calories behind in her caloric needs for the day (burning 60 RMR calories per hour over 12 hours and 50 exercise calories plus 600 calories of food during breakfast leaves the user with a 170 calorie deficit). If the user maintains this deficit through the day, she will be on track to meet longer-term weight loss goals. Thus, by including users' various metabolic rates, their food intake, and their exercise regimen, the health system can give users real-time information about how well they are tracking to a daily caloric goal.

For example, a cell phone may be connected via a wired link, or through a short range wireless link, to a heart rate or other biometric monitor on the user's chest, wrist or other area. The cell phone, with appropriate client software stored on the phone, receives signals from the monitoring device and displays to the user vitals, such as heart rate and performance criteria to help the user achieve a desired fitness goal (e.g., to burn fat without burning muscle). If the cell phone also included an audio playback device, such as an MP3 player, then the user could also simultaneously listen to audio recordings. This audio content could come from the health system. It may be a motivational message chosen, for example by a health care worker to support the user's determination to reach or maintain her health goals. The audio content may include music chosen by the user (or by someone else for the user) to listen to while the user exercises. If the user's handheld device supports the playback of video, the user may be able to play a workout video that can guide her exercise routine.

Alternatively, users may want to listen to or watch health-related content during free moments through the day. They may watch video content that will support their motivation to achieve and maintain her health goals. Alternatively, they may watch video content that teaches them about health conditions they may have, exercise techniques, healthy cooking they want to learn about, or other health-related video content. A health care provider who is aware of a user's specific health concerns may choose video or audio content to educate the user about her condition and about important self-care habits. The health system thus integrates health-related content from a variety of sources and brings it to the user in a format that is convenient and easy to use.

As noted above, the health system can provide for various customized diets for the user. For example, the health system can provide a 7-day, balanced, low calorie menu to guide the user, where the menu has preset fat/protein/carbohydrate items with associated caloric values. The menus would also include and be specific to certain food needs, or different physical conditions, such as to compensate for high cholesterol, diabetes, heart disease, kosher, vegetarian, and so forth. Thus, a user can consume a desired amount and type of foods per day, to thereby stick to certain diets, such as a high protein/no carbohydrate diet, and so forth. The health system may choose advertisements based on a user's diet choices and information available through the Healthy Living City Guide. For example, the health system might choose to show a user on a low-carbohydrate diet an advertisement for a low-carbohydrate food that is on sale at a local supermarket. The health system might choose to show a user on a high-protein diet an advertisement for a nearby steak restaurant. The health system can allow a user to download meal suggestions from a nutrionist, possibly including recipes or other types of meal suggestions.

These diet menus may be associated with guides that can provide a suggested set menu of calories/protein/carbohydrates/fat that the user should consume if the user were training for a marathon, triathlon, or other set athletic event. Further, the health system could provide a training schedule for the user to achieve a certain athletic goal, including target heart rates, distances, and associated menu for each day of the training schedule.

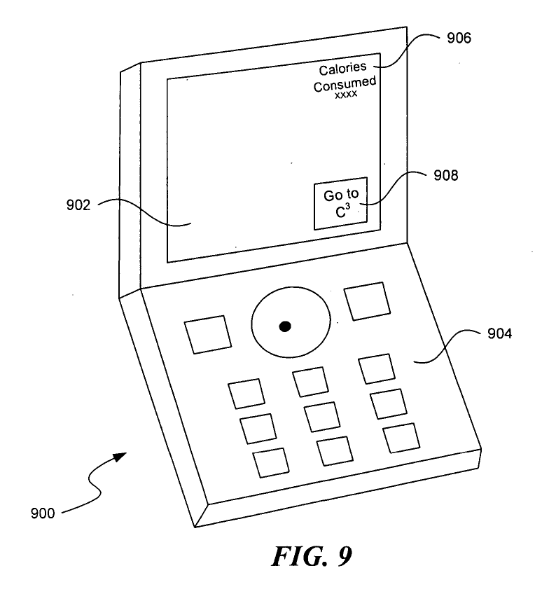

The health system can prompt the user to input foods consumed during the day, and display a running total of calories consumed for a given day. FIG. 9 is an example of a simple display screen 902 on a cell phone 900. As shown, the phone also includes a standard input device or keypad 904. Calories consumed is shown in an upper right corner 906 of the display 902. As shown, the running total of daily calories consumed 906 is featured on the desktop of the phone, and thus is always displayed to the user throughout the day as a reminder. A lower right icon 908 allows the user to readily click on that icon to access a simple menu for providing much of the functionality described above. For example, clicking on the icon 908 allows the user to be provided with a simple two or three level menu tree of options. At the first level, choices could include:
1. Monitoring heart rate and accessing heart rate functions;
2. Accessing a database of calories for food items, drink items, and menus;
3. Accessing a meal timer or calendar; and
4. Accessing specific Internet sites and location-specific data.

By accessing, for example, the calorie database option, the user could then be presented with a simple alphabetical list of food items to scroll through, or to randomly access by punching in the first few letters of a food item using the keypad 904. Thereafter, the user inputs a type and/or size of portion for the selected food item to be added to the daily calories consumed running total 906. In particular, in this embodiment, the menu structure is very simple. Thus, the calorie counter database can be accessed like a standard address book common on many cell phones.

As another example, if the health system recognizes that the user is in a new location, such as a new city, the user may access the fourth item from the menu and then be given options to access local restaurants, health food stores or gyms in that city. Each of these establishments may subscribe to the health system to thereby be listed, and possibly pay an additional fee to be listed first. By selecting one of the listed establishments, the phone may then display an address, phone number, and map of the selected establishment. For example, the GPS or other location determining technology of the phone can determine that the phone has been turned on or is otherwise currently located in a new city. If the user selected a particular restaurant after having traveled to Boston, then a list of items on the menu at that restaurant may be displayed, along with associated calories for each item. This can help the user not only locate desirable restaurants in a new city, but also help identify which items on the restaurant's menu to order.

Thus, the health system may use location/GPS technology to provide further features. For example, when a user is seeking a restaurant to eat at, the user can access the Healthy Living City Guide to find restaurants close to the user's physical location, along with information about food available at these restaurants so the user can determine which restaurants are compatible with the user's health goals. Similarly, information about health clubs or outdoor physical activities may also be provided based on a user location. In both instances (seeking food or exercise), advertising may be available for restaurants, health clubs or other entities. The location may be determined based on built-in GPS technology, or based on cell site information in a cellular network. In this way, the cell phone can provides access to features of the Healthy Living City Guide where ever the user may be.

Further, the GPS unit or other location determining technology associated with the cell phone can estimate speed and distance of the user while running, which can help estimate number of calories burned during a given workout.

The health system may also permit the user to create bins or folders of information beyond simply a running total of daily calories consumed. This information can then be shared with others automatically, such as by sending not only daily calories consumed, but also exercise information (including heart rate) to a doctor or personal trainer. Information may also be shared with the user's group of friends or colleagues, where the user has created a predetermined group who can receive such information.

Along these lines, workouts may be arranged in a community, allowing for competition, support and positive reinforcement within the community. Military units often train as a group to provide support for individual members. Similarly, groups of individuals at a gym, within a neighborhood, or at a company, for example, may have a group exercise plan which is monitored within the health system and for which information is made available to group members through their phones. Additionally, this can be extended to allow instructors or trainers to monitor individual progress, and may similarly be used to provide access to physicians and other advisors (e.g. dieticians, coaches) for information on the individual user. Moreover, social networking may be involved, such that disparate groups of individuals with a common link socially may be involved in reinforcing each other's attempts to achieve or maintain physical fitness.

Communities or groups may be further enhanced through use of messaging capabilities. Text messaging and SMS protocols allow for simple messages to be propagated through a wireless phone system in a simple manner. Thus, users may be alerted to a scheduled group activity through messages sent at predetermined intervals. Alternatively, messages may be sent to provide encouragement, or to provide updates on progress, allowing users to keep in mind their goals at times when exercise is not normally on their minds. The health system can send messages containing short workout videos that users can do at their desks, or while commuting. The health system can also use messages to provide timely news about health-related topics such as new research about the benefits of exercise, healthy living choices, and mental and emotional wellness. Additionally, discussions within a community may be carried out using text messages. Moreover, messages may be automatically generated when a user exceeds or meets preset thresholds, whether for achieving goals or falling behind on goals.

As one may expect, this system may be provided on a subscription basis to individuals or groups. Thus, an individual could subscribe on an ongoing basis, or during times when the individual seeks to achieve certain fitness or health goals. Similarly, fitness companies (e.g. health clubs) may subscribe on a group basis and offer the service as a benefit or value-add for members (whether charging a premium or not). Likewise, hospitals or insurance/health maintenance organizations may subscribe on a group basis and offer the health system to patients or clients. Alternatively, phone providers (service providers) may offer the service to subscribers. Much of the technology described herein can be provided by a wireless service provider (e.g., Cingular®, T-Mobile®), or could be factory installed by a cell phone manufacturer (e.g., Nokia®, Motorola®).

Another feature that may be provided is reporting of results. This may occur in electronic form, such as through email sent to a user or authorized persons working with a user. Thus, a doctor or coach may receive reports electronically, along with the user. Additionally, reports may be sent in paper form, or printed for the user in some circumstances. These reports may be periodic in nature, providing a regular check on progress, or may be provided on demand, for example. The reports may provide information on a variety of different metrics, such as food consumption, exercise, breakdowns of subcategories of consumption or exercise, or some combination of metrics. When reports are provided electronically, one form the reports may take is that of a shopping cart—showing different foods in a graphical manner, for example. Additionally, graphical representations such as pie or bar charts may be provided, too. Moreover, reports may provide, total, percentage, proportion, variation over time, or other representations of data, whether in a textual or graphical representation.

In an alternative embodiment, the health system may incorporate virtual reality features to display an animated or computer-generated personal instructor who can help motivate the user and remind him or her of things to help themselves in the environment, such as walking or biking to work, recycling, choosing one food over another (e.g., nonfat yogurt over sour cream), etc. Such a virtual reality or animated assistant can be displayed on any suitable display, and may have appropriate and associated audio generated for the user.

The health system can also link to the Internet and allow the user to access specific health or fitness sites, to thereby obtain current fitness news, gather information regarding desired health or fitness plans or goals, and so forth.

By providing an integrated system that fully utilizes handheld and other computing devices, users can manage their health wherever they are. The Healthy Living City Guide and related functionality allow users to collect information about their diet and exercise choices and receive real-time feedback on those choices. By using location-awareness, the health system provides users with quick access to the information they need to make wise health-related choices and take advantage of local health-related resources even when they are in an unfamiliar city. The deep integration provided by this system allows users to improve coordination with their health care providers regarding many aspects of their lifestyle choices. In addition to providing health care workers with insights into the daily life of their patients, the health system also enables the health care providers to have day-to-day impact on improving the health of their patients. The integration of many health-related features users need in a seamlessly integrated health system makes it significantly easier to enable the users to attain and maintain optimal health.

In some embodiments, the health system's Healthy Living City Guide feature receives an indication of a geographical location, such as via a GPS signal or indication of a telephone area code. The health system can then display a list of health-related resources in categorical form, such as retail establishments near the indicated geographical location. Examples of health-related resource categories include restaurants, grocery stores, gyms, spas, trainers, Pilates centers, spinning centers, yoga centers, chiropractors, nutritionists, healers, doctors, acupuncturists, and so forth. When the user selects a category, specific health-related resources and identifying information may appear, such as names, their addresses, telephone numbers, hours of operation, directions, and so forth. As an example, when a restaurant is selected, the identifying information may appear in addition to a link to a menu listing foods the restaurant may serve. The menu may additionally contain information about served dishes and their caloric values. The user can select a particular dish or menu item to indicate that the user has consumed it. When the user performs this action, the health system can count the associated caloric value as having been consumed by the user. In various embodiments, the health system is capable of providing health-related information for one or more geographical areas, for all cities in the world, etc. Similarly, the health system can subtract expended calories when the user selects an activity at a gym and specifies the amount of time that the activity was performed.

In some embodiments, the health system sends a periodic reminder, such as a daily reminder, weekly reminder, monthly reminder, yearly reminder, and so forth. These periodic reminders remind users to perform some healthy task (e.g., exercise), provide motivational messages or updated information on health and fitness, or display advertisements. In various embodiments, the reminders and advertisements can be sent as alerts. The advertisements can be textual, graphical, aural, or audiovisual. In some embodiments, the periodic reminder is broadcast to many users. As an example, users may be able to access a Daily Reminder feature, which is a live Mobile Television (wireless broadband) broadcast, every hour on the hour for one minute, reminding the user of key daily tasks, as well as updated news on health and fitness. In addition, The Daily Reminder can be used to send SMS CELL BROADCAST (SMS-CB) advertisements simultaneously to potentially millions of mobile handsets in specified areas, ranging from a single cell to an entire network. This feature will enable the health system to deliver germane information to multiple users nearly simultaneously.

In some embodiments, the health system receives (or monitors) the user's heart rate and computes the number of calories the user has burned based on the heart rate. The health system can deduct the expended calories from the user's calorie count (e.g., caloric goal).

In some embodiments, the health system receives various health-related information from users, such as their age (or date of birth), height, weight, sex, and so forth. The health system may employ this information in its calculations and recommendations, such as for foods.

These and other changes can be made to the invention in light of the above Detailed Description. While the above description describes certain embodiments of the invention, and describes the best mode contemplated, no matter how detailed the above appears in text, the invention can be practiced in many ways. Details of the health guidance system may vary considerably in its implementation details, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

I claim:

1. A method performed by a computer system for enabling a user to manage the user's health by using a mobile computing device that communicates with a health system, the method comprising:
receiving a caloric request from the user, the caloric request indicating an amount of calories that the user desires to consume during a first time interval;
identifying a resting metabolic rate for the user, the resting metabolic rate indicating a theoretical number of calories the user consumes during a second time interval;
receiving information relating to physical activity performed by the user beginning at the start of the first time interval and ending at a specified time;
computing an expended number of calories based on the physical activity performed by the user and resting metabolic rate by retrieving information from a database located on the mobile computing device, the database containing a correspondence between foods and calories;
receiving information relating to food consumed by the user beginning at the start of the first time interval and ending at the specified time;
computing a consumed number of calories based on the food consumed by the user;
determining a status for the user based on whether the user is to consume calories or expend calories by analyzing the computed expended number of calories and the computed consumed number of calories;
receiving geographic location information identifying a geographic location at which the user is located;
sending an alert to the user based on the status for the user and the received geographic location information.

2. The method of claim 1 wherein the determining comprises:
computing a difference between the computed expended number of calories and the computed consumed number of calories; and
when the computed consumed number of calories exceeds the computed expended number of calories, the method further comprises identifying a recommended physical activity to the user.

3. The method of claim 1 wherein the first time interval is a twenty-four hour period and the second time interval is an hour.

4. The method of claim 1 wherein the computing an expended number of calories includes retrieving information from a database located on a server computing system.

5. The method of claim 1 wherein sending the alert includes sending a motivational message to the user.

6. The method of claim 1 wherein sending the alert includes sending an advertisement to the user.

7. The method of claim 1 wherein sending the alert includes sending a location of a retail establishment that is located near the user's identified geographic location.

8. The method of claim 1 wherein sending the alert includes sending a location of a restaurant serving healthy food that is located near the user's identified geographic location.

9. The method of claim 1 wherein sending the alert includes sending a location of a health club that is located near the user's identified geographic location.

10. The method of claim 1 further comprising sending a scheduled alert to the user that is unrelated to the alert that is sent to the user based on the status for the user.

11. A computer-computer-readable medium storing computer-executable instructions that, when executed, perform a method for enabling a user to manage the user's health by using a computing device that communicates with a health system implemented by the computer system, the method comprising:
receiving a caloric request from the user, the caloric request indicating an amount of calories that the user desires to consume during a first time interval;
identifying a resting metabolic rate for the user, the resting metabolic rate indicating a theoretical number of calories the user consumes during a second time interval;
receiving information relating to physical activity performed by the user beginning at the start of the first time interval and ending at a specified time;
computing an expended number of calories based on the physical activity performed by the user and resting metabolic rate;
receiving information relating to food consumed by the user beginning at the start of the first time interval and ending at the specified time;
computing a consumed number of calories based on the food consumed by the user;
determining a status for the user based on whether the user is to consume calories or expend calories by analyzing the computed expended number of calories and the computed consumed number of calories; and
sending an alert to the user based on the status for the user.

12. The computer-computer-readable medium of claim 11 further comprising receiving geographic location information identifying a geographic location at which the user is located.

13. The computer-computer-readable medium of claim 11 further comprising sending a scheduled alert to the user that is unrelated to the alert that is sent to the user based on the status for the user and that provides an indication of the computed expended number of calories or the computed consumed number of calories.

14. The computer-computer-readable medium of claim 11 wherein the receiving information relating to physical activity performed by the user includes receiving the user's heart rate and the expended number of calories is computed based at least on the user's heart rate.

15. A system for enabling a user to manage the user's health, comprising:
a component that receives a caloric request and a resting metabolic rate indicating a theoretical number of calories the user consumes during a second time interval, the caloric request indicating an amount of calories that the user desires to consume during a first time interval;
a component that computes an expended number of calories based on the user's resting metabolic rate and physical activity performed by the user;
a component that computes a consumed number of calories based on food the user consumes beginning at the start of the first time interval and ending at a specified time;
a component that determines a status for the user based on whether the user is to consume calories or expend calories by analyzing the computed expended number of calories and the computed consumed number of calories; and
a component that sends an alert to the user based on the status for the user.

16. The system of claim 15 further comprising a sensor component that monitors physical activity on an exercise equipment and transmits an indication of the physical activity to the component that computes an expended number of calories.

17. The system of claim 15 further comprising a biometric sensor component that monitors a physiological condition of the user and transmits an indication of the physiological condition to the component that computes an expended number of calories.

18. The system of claim 15 further comprising a sensor component that monitors physical activity performed by the user and wirelessly transmits an indication of the physical activity to the component that computes an expended number of calories.

19. The system of claim 15 further comprising a display unit that displays sent alerts and menus to the user, the menus identifying foods that the user can consume based on the received caloric request.

20. The system of claim 15 further comprising a display unit that displays advertisements to the user, the advertisements based on diet choices made by the user, the diet choices associated with the received caloric request.

21. A method performed by a computer system for providing a healthy city living guide, comprising:
receiving a caloric goal from a user, the caloric request indicating an amount of calories that the user desires to consume during a twenty-four hour time interval;
receiving a resting metabolic rate for the user, the resting metabolic rate indicating a theoretical number of calories the user consumes during a one-hour time interval;
receiving information relating to physical activity performed by the user and food consumed by the user;
computing a caloric difference based on the received caloric goal, the received resting metabolic rate for the user, and the received information relating to physical activity performed by the user and food consumed by the user; and
when the computed caloric difference indicates that the caloric goal will not be met, sending an alert to the user wherein the alert comprises a multimedia content relating to physical activity, a menu selection identifying a food, a meeting location for physical activity, or a message from a health professional.

22. The method of claim 21 wherein the meeting location provided by the alert identifies a meeting location at which other users will be engaged in a physical activity identified as beneficial to the user's caloric goal.

23. The method of claim 21 further comprising sending a message to a health professional indicating the computed caloric difference.

24. The method of claim 21 further comprising sending a message to a health professional indicating the physical activity performed by the user or the food consumed by the user.

25. The method of claim 21 further comprising providing a news article to the user relating to the caloric goal.
